(19) United States
(12) Patent Application Publication
(10) Pub. No.: US 2026/0298864 A1
(43) Pub. Date: Oct. 1, 2026

(54) ETRS-Based Biosensing Method and System

(71) Applicant: The University of Hong Kong, Hong Kong (HK)

(72) Inventors: Shiming ZHANG, Hong Kong (HK); Xinyu TIAN, Hong Kong (HK); Jing BAI, Hong Kong (HK); Dingyao LIU, Hong Kong (HK)

(21) Appl. No.: 19/634,136

(22) Filed: Mar. 31, 2026

Related U.S. Application Data
(60) Provisional application No. 63/780,482, filed on Mar. 31, 2025.

(51) Int. Cl. G01N 27/327; G16B 40/10

(52) U.S. Cl. CPC G01N 27/3275; G16B 40/10

(57) ABSTRACT

The present invention provides and electrochemical transistor resonance spectroscopy (ETRS)-based biosensing system and method. The system comprises: a biosensing module configured to sense one or more target bioanalytes and stimulated under a plurality of frequency-amplitude combinations provided by a signal generator; a data processor configured to receive and process a time-domain sensing signal from the biosensing module to predict concentrations of the one or more target bioanalytes. The data processor is implemented with one or more artificial neural networks and configured to use the one or more artificial neural networks to analyze the time-domain sensing signal with a ETRS approach. The present invention enables a limit of detection (LoD) as low as 1 fM, that is up to six orders of magnitude lower than conventional electrochemical methods.

### CROSS-REFERENCE TO RELATED APPLICATIONS

The present application claims priority from the U.S. Provisional Patent Application No. 63/780,482 filed 31 Mar. 2025, and the disclosure of which is incorporated herein by reference in its entirety.

### REFERENCE TO SEQUENCE DISCLOSURE

The sequence listing file under the file name “P3501US01_Sequence_listing.xml” submitted in ST.26 XML file format with a file size of 3,847 bytes created on Jun. 11, 2026 is incorporated herein by reference.

### FIELD OF THE INVENTION

The present invention generally relates to biosensing technologies. More specifically the present invention relates to organic electrochemical transistor (OECT) biosensing method and system based on electrochemical transistor resonance spectroscopy (ETRS).

### BACKGROUND OF THE INVENTION

Wearable sensors have gained significant attention with the rise of smartphones and mobile devices due to their critical role in enabling pervasive health monitoring. These devices provide valuable insights into individual health and performance while serving as key tools for collecting datasets essential for building large health models in enabling digital health and AI medicine. Early developments in wearable technology focused on physical sensors that monitored movement and vital signs, such as steps taken, calories burned, or heart rate. In recent years, wearable devices have rapidly evolved, shifting from tracking easily accessible physical activities to addressing critical healthcare challenges, such as diabetes management and remote monitoring of the elderly. To meet the above demands, a new wave of research has emerged, focusing on the development of wearable biosensors capable of monitoring biochemical signals. These devices integrate biological recognition elements-such as enzymes, antibodies, aptamers or cell receptors-into their operation (such as amperometric, cyclic voltammetry (CV), differential pulse voltammetry (DPV), square-wave voltammetry (SWV)). The potential of wearable biosensors is evident from the growing number of proof-of-concept studies, with some devices, like continuous glucose monitoring systems (CGMs), already commercialized. However, advancing this field places an urgent demand on new sensor technologies. Most clinically relevant bioanalytes exist at extremely low concentrations (nanomolar to femtomolar), posing significant challenges for current electrochemical methods. For example, glucose, as detected by commercial CGMs, exists in concentrations between 1-10 mM, but bioanalytes such as insulin are typically in the picomolar range, while COVID-19 biomarkers are in the femtomolar range. Detecting these molecules at such low concentrations requires improved sensor technologies.

OECTs show great promise for biosensing applications due to their ability to amplify signals directly within the sensor. The gate electrode of an OECT detects electrochemical signals, while the transistor amplifies the signal, achieving amplification factors of up to 10000, among the highest for transistor-based biosensors. Additionally, the redox-controlled working mechanism enables OECTs to operate at bio-level low voltages (millivolts) while maintaining high amplification capabilities, making them highly suitable for interfacing with living systems.

Due to the above advantages, OECTs are being increasingly used in wearable and implantable biosensing applications. However, current OECT-based sensors have delivered mixed results when detecting biochemical signals. Early research efforts focused on increasing the amplification ratio (transconductance, Gm) to improve sensitivity, while others analyzed shifts in transfer curves. Despite these progresses, there is still no unified method to demonstrate the ultimate sensitivity of OECTs for specific bioanalytes or to standardize their performance against conventional three-electrode electrochemical systems. Addressing this gap is critical for enabling fair evaluation, large-scale validation, and commercialization of OECT-based biosensors.

Conventional frequency-dependent electrochemical techniques, such as EIS, are constrained by their reliance on linearity. These methods require small signals, leading to poor SNR. Additionally, the behavior of the electrochemical double layer is inherently nonlinear, and linear methods fail to capture essential nonlinear kinetic information directly tied to molecular detection, ultimately limiting detection accuracy.

Previously reported OECT biosensors, analyzed through transfer curves, inherently operate in a nonlinear detection mode. However, these approaches excessively rely on overly simplistic mathematical models, which restrict their full potential.

### SUMMARY OF THE INVENTION

To overcome the abovesaid limitation, the present invention introduces an ETRS technique based on an integrated sensing concept that consolidates diverse OECT characterization methods into a single process. Based on the ETRS technique, a comprehensive frequency scan is performed and a complex high-dimensional correlation analysis on the harmonic responses is performed using artificial neuron networks (ANN). These ANNs, pre-trained on extensive experimental datasets, enable the analysis of complex harmonic patterns in the OECT drain-source current data and reveal deep correlations between the harmonic patterns and bioanalyte concentrations.

In accordance with a first aspect of the present invention, an ETRS-based OECT biosensing system is provide. The system comprises: a biosensing module configured to sense one or more target bioanalytes and generate a time-domain sensing signal corresponding to concentrations of the one or more target bioanalytes; a signal generator coupled to the biosensing module and configured to generate a stimulating signal for application to the biosensing module, and further configured to co-scan a frequency and an amplitude of the stimulating signal over an operating frequency range and an operating amplitude range, respectively, such that the biosensing module is stimulated under a plurality of frequency-amplitude combinations; and a data processor coupled to the biosensing module and configured to receive and process the time-domain sensing signal to estimate the concentrations of the one or more target bioanalytes. The data processor is implemented with one or more artificial neural networks and is configured to: receive the time-domain sensing signal from the biosensing module in response to the plurality of frequency-amplitude combinations of the stimulating signal; transform the time-domain sensing signal into a frequency-domain sensing signal; analyze, using the one or more artificial neural networks and an ETRS approach, both the time-domain sensing signal and the frequency-domain sensing signal to identify one or more optimal stimulation conditions, each optimal stimulation condition including an operating frequency and an operating amplitude associated with a respective target bioanalyte; extract one or more resonance characteristics of the biosensing module from the time-domain sensing signal and the frequency-domain sensing signal, the one or more resonance characteristics; and predict respective concentrations of the one or more target bioanalytes based on the one or more identified optimal stimulation conditions and the one or more resonance characteristics.

In accordance with a second aspect of the present invention, an ETRS-based OECT biosensing method is provided. The method comprises: generating, by a signal generator coupled to the biosensing module, a stimulating signal for application to the biosensing module; co-scanning, by the signal generator, a frequency and an amplitude of the stimulating signal over an operating frequency range and an operating amplitude range, respectively, such that the biosensing module is stimulated under a plurality of frequency-amplitude combinations; sensing, by a biosensing module, with one or more target bioanalytes; generating, by the biosensing module, a time-domain sensing signal corresponding to concentrations of the one or more target bioanalytes; receiving, by a data processor coupled to the biosensing module, the time-domain sensing signal from the biosensing module in response to the plurality of frequency-amplitude combinations of the stimulating signal; transforming, by the data processor, the time-domain sensing signal into a frequency-domain sensing signal; analyzing, by the data processor implemented with one or more artificial neural networks and using an ETRS approach, both the time-domain sensing signal and the frequency-domain sensing signal to identify one or more optimal stimulation conditions, each optimal stimulation condition including an operating frequency and an operating amplitude associated with a respective target bioanalyte; extracting, by the data processor, one or more resonance characteristics of the biosensing module from the time-domain sensing signal and the frequency-domain sensing signal; and predicting, by the data processor, respective concentrations of the one or more target bioanalytes based on the one or more identified optimal stimulation conditions and the one or more resonance characteristics.

The ETRS technique can reflect the maximum sensitivity achievable by OECT-based sensors. This is achieved by scanning a gate-stimulating voltage across a wide range of frequencies to identify a resonance frequency specific to each bioanalyte. This resonance represents the optimal synergy between the detecting electrodes and the OECT based sensor, enabling a limit of detection (LoD) as low as 1 fM (e.g., for Na⁺), up to six orders of magnitude lower than conventional three-electrode electrochemical methods.

The increased sensitivity stems from: i) the in-sensor-coupled transistor, which amplifies the detected signals at their origin, thus improving the signal-to-noise ratio (SNR); and ii) ANN-enabled numerical frequency-domain analysis, which reflects more information about the dynamics of the sensory electrochemical double-layer that is otherwise not possible through existing temporal analytical methods.

It is demonstrated that the ETRS platform can be applied in other common scenarios where electrochemical sensors are employed, exemplified by its ability to overcome the sensitivity limitations of current biochips in multimodal sensing.

### BRIEF DESCRIPTION OF THE DRAWINGS

Embodiments of the invention are described in more details hereinafter with reference to the drawings, in which:

FIG. 1 illustrates a workflow of a ETRS process (bottom) in accordance with various aspects of the present invention and an illustrative comparison against conventional electrochemical impedance spectroscopy (EIS) (top).

FIG. 2A illustrates schematic of Ids frequency analysis using the Fast Fourier Transform.

FIGS. 2B to 2G show evaluation results of signal reconstruction accuracy using an increasing number of harmonic components, from the fundamental (1^{st}) to the 6^{th} harmonic, respectively.

FIG. 2H shows quantitative assessment of the reconstruction error.

FIG. 3A shows time-domain representation of the input voltage (V_{g}, top) and corresponding output current (I_{ds}, bottom) and their use for detecting biomarkers at different input frequencies (0.1 Hz to 10 kHz).

FIG. 3B show frequency-domain representation of the corresponding output current I_{ds} using FFT, revealing the harmonic components associated with each input frequency.

FIG. 3C shows frequency-dependent response in ETRS for a fixed bioanalyte concentration, plotting the magnitude and phase of the I_{ds} across input frequencies from 0.1 Hz to 10 kHz, illustrating how higher-order harmonics enhance the fidelity of the measured signal.

FIG. 3D shows the THD calculated at different input frequencies, showing how THD varies with frequency input, indicating the distortion levels in the ECT output current.

FIG. 4A shows analytical simulation results of absolute change of voltage drop on channel with the increased input frequency. In short, under the resonance frequency; FIG. 4B shows relative change of voltage drop on channel with the increased input frequency; and FIG. 4C shows concentration changes of the biomarker with the increased input frequency.

FIG. 5 illustrates a process flowchart of an ETRS-based OECT biosensing method for monitoring concentrations of one or more bioanalytes in accordance with the present invention.

FIG. 6A shows a non-FFT approach (M1), which can direct input of time-domain current signal; FIG. 6B shows a FFT approach (M2), which can input harmonic information derived from FFT of the time-domain signal; and FIG. 6C shows a combined approach (M3), which used a leaner architecture combining both time-domain signal and FFT-derived harmonic information.

FIG. 7 shows comparison of computational efficiency in terms of floating point operations per second (kFLOPs) and accuracy among the three approaches.

FIG. 8 illustrates a schematic diagram of a wireless ETRS-based OECT biosensing system in accordance with one embodiment of the present invention.

FIGS. 9A and 9B illustrate a top view and a bottom view of a prototype of the biosensing system, respectively.

FIG. 10 illustrates experimental data that shows voltage input and current measurement at different frequencies.

FIG. 11 illustrates a schematic diagram of an OECT biosensor in accordance with one embodiment of the present invention.

FIG. 12 illustrates impedance mathematical models for various circuit components of the OECT biosensor.

FIG. 13A illustrates operation of the ETRS-based OECT biosensor; and FIG. 13B shows entire ETRS spectrum correspondingly generated by the ETRS-based OECT biosensor.

FIG. 14 is schematic depicting the layout of the OECT biochip, featuring an array of OECT sensor units.

FIG. 15 is an exploded view of the OECT biochip assembly.

FIG. 16 illustrates an example fabrication flow for the OECT biochip.

FIG. 17 shows a schematic of an ionic biosensor modified from the OECT biosensor.

FIG. 18 illustrates steps for modification of an ISM on the channel of the OECT sensor to fabricate an OECT Na⁺ sensor.

FIG. 19 shows a schematic structure of an ion selective OECT biosensor.

FIG. 20 shows transfer characteristics of a conventional OECT before and after coating an ISM, and the device with PSSNa as the inner electrolyte.

FIG. 21 shows comparison of current response to Na⁺, K⁺ and Ca²⁺ at different concentration at fixed V_{g}=0 V and V_{ds}=−0.4 V.

FIG. 22 shows signals extracted from ETRS spectrum for sensing Na⁺ ions at different frequencies (0.1 Hz, 600 Hz, 10 kHz), which shows superior sensitivity at the resonance frequency (600 Hz).

FIG. 23 illustrates the LoD of the ETRS-based biosensor for sensing Na⁺ ions over other competing technologies.

FIG. 24 shows a schematic of an aptamer-based biosensor modified from the OECT biosensor.

FIG. 25 is a schematic representation of the functionalization process for the aptamer-based biosensor.

FIG. 26 illustrates sequences of the DNA aptamers used for target detection, including the insulin end probe and corresponding predicted 2D structures by Mfold.

FIG. 27 illustrates impedance changes observed on the electrode surface throughout the functionalization process, illustrating the transition from bare gold to the aptamer-modified surface and subsequent blocking with 6-MCH.

FIG. 28 shows verification of successful modification using Square Wave Voltammetry.

FIG. 29 shows a schematic of an enzyme-based biosensor modified from the OECT biosensor.

FIG. 30 shows steps for the preparation of enzyme-modified gate electrode of the OECT biosensor to form the enzyme-based OECT biosensor.

FIG. 31 shows the electron transfer process on an enzyme-modified electrode of a glucose biosensor in the presence of glucose.

FIG. 32 shows cyclic voltammetry curve of the modified electrode in 0.1 M PBS. The presence of redox peak is ascribed to the ferrocene, which is the electron transfer mediator.

FIG. 33 shows cyclic voltammetry curve of the modified electrode before and after adding 50 mM glucose.

FIG. 34 shows response of OECT glucose sensors to potential interfering substances, including ascorbic acid (AA) and urea (n=5). Data are presented as means±SD. PBS, phosphate-buffered saline.

FIG. 35 shows an integrated ETRS-based biosensing system integrated with an ionic biosensing unit, a enzymes-based biosensing unit, an aptamer-base biosensing unit and a cortisol sensing unit.

FIGS. 36A to 36D illustrate the LoD of different aptamer-based OECT biosensors detecting cortisol, insulin, and SARS-CoV-2 spike protein and glucose, respectively.

FIGS. 37A to 37C illustrate the signal change of the aptamer-based OECT biosensor when detecting different non-specific targets.

FIG. 38 shows characterization schematics of OECT device of the present invention and a conventional EC device.

FIG. 39 shows SNR comparison under simulated noise conditions.

FIG. 40 shows an example implementation of an integrated wearable ETRS platform for the detection of low-concentration bioanalytes.

FIG. 41 illustrates the sensing mechanism of the OECT biosensor. Target molecules (e.g., cortisol) are captured by the aptamer, and the signals are amplified by the OECT.

FIG. 42 shows an ETRS spectrum generated after a frequency scan.

FIG. 43 shows experimental data of the cortisol concentration signals extracted from the ETRS spectrum.

### DETAILED DESCRIPTIONS

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

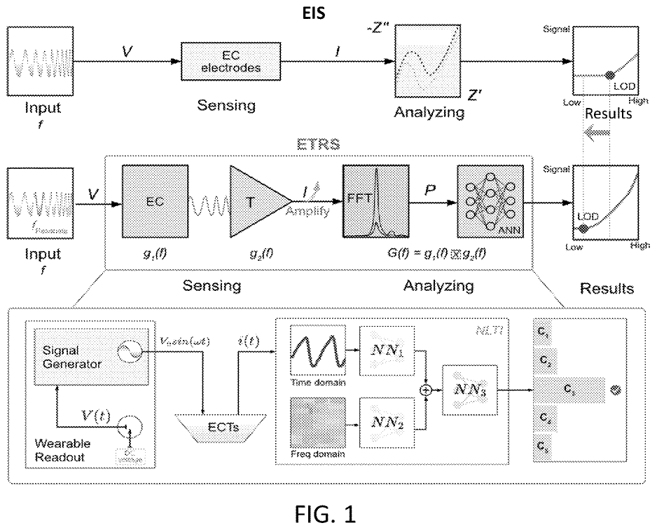

FIG. 1 illustrates a workflow of a ETRS process (bottom) in accordance with various aspects of the present invention and an illustrative comparison against conventional electrochemical impedance spectroscopy (EIS) (top). The ETRS process features a large input signal and uniquely integrates a transistor (T) into the sensing circuit to amplify the signal. The amplification ratio achieves its maximum value at a specific resonance frequency (f_{Resonate}), which indicates the optimal synergy between the sensing electrode and the transistor amplifier. An ANN is employed for pattern recognition and quantification of the amplified non-linear signals after Fast Fourier Transform (FFT).

The ETRS process is analogues to a non-linear EIS process, but uniquely integrates with the electrochemical transistor for in-sensor amplification. Non-linear EIS, unlike conventional EIS, applies larger amplitude stimulation signals and does not adhere to linear response constraints, allowing for the extraction of richer dynamic information about the electrochemical system, essential to improve the sensitivity.

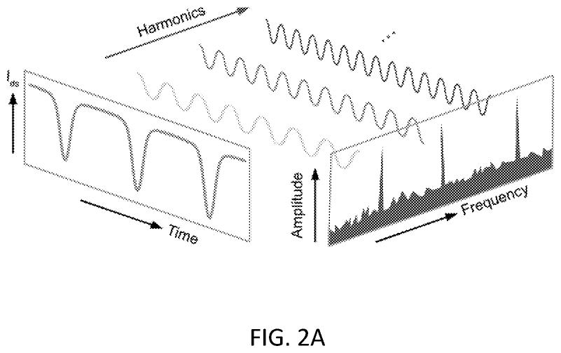

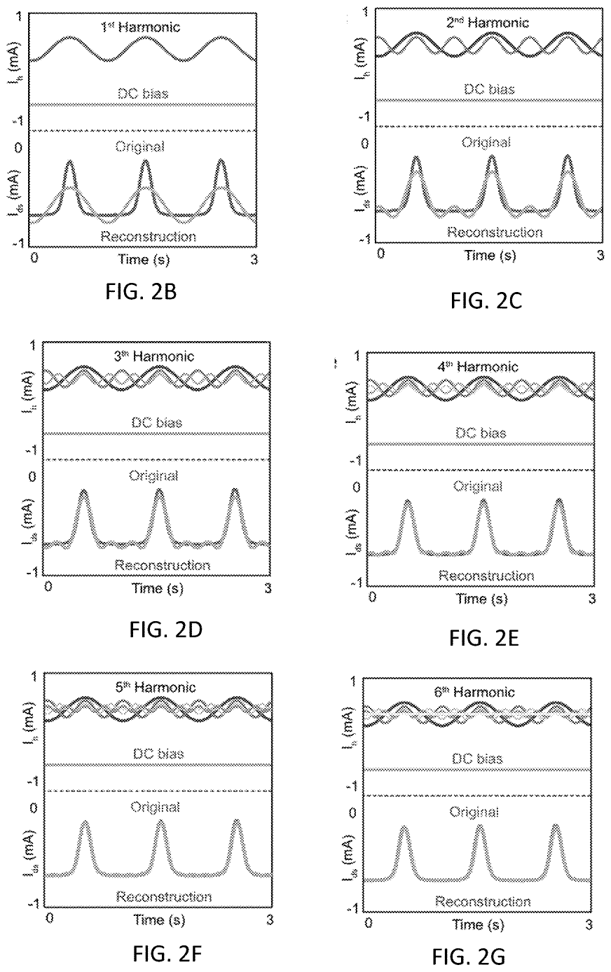

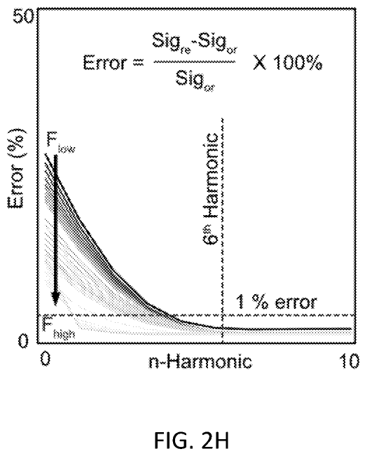

In the frequency domain, higher-dimensional information, such as the distribution of high-order harmonics and their power densities are obtained. FIG. 2A illustrates schematic of Ids frequency analysis using the FFT. The FFT can produce numerous secondary harmonics with minimal informational content. Therefore, it is essential to identify the primary harmonic components for further analysis.

FIGS. 2B to 2G show evaluation results of signal reconstruction accuracy using an increasing number of harmonic components, from the fundamental (1^{st}) to the 6^{th} harmonic, respectively. In the FIGS. 2B to 2G, the top panels show the DC bias and the respective harmonic waveforms used for reconstruction; the lower panels compare the original measured signal with the reconstructed waveform. FIG. 2H shows quantitative assessment of the reconstruction error, defined as IRMS__{reconstructed}−RMS__{original} 1/RMS__{original}, demonstrates that including up to the 6^{th} harmonic reduces the error below 1%. Thus, only the first six harmonics need to be further analyzed during the ETRS.

It is also noted that these harmonic distributions and power densities are significantly more sensitive to fluctuations in the electrochemical system, such as changes in bioanalyte concentration.

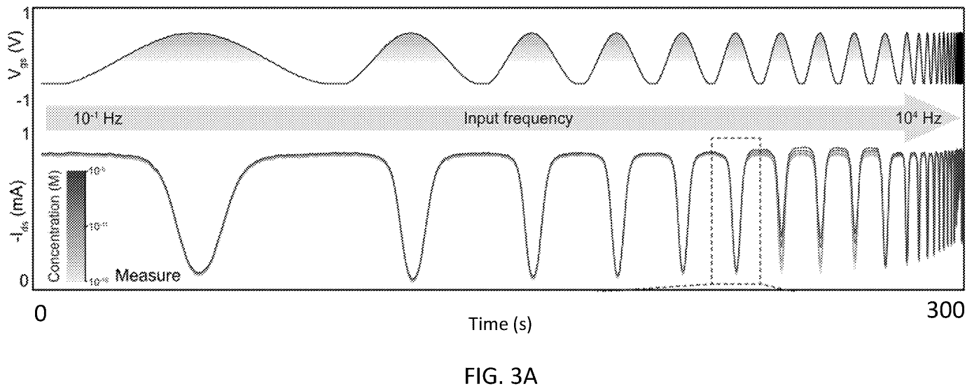

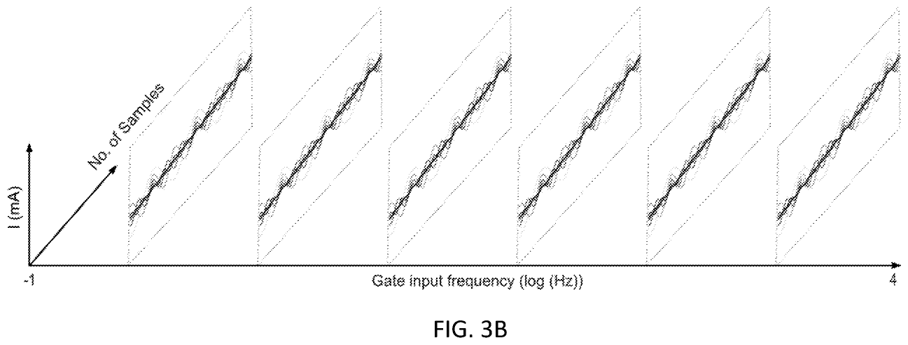

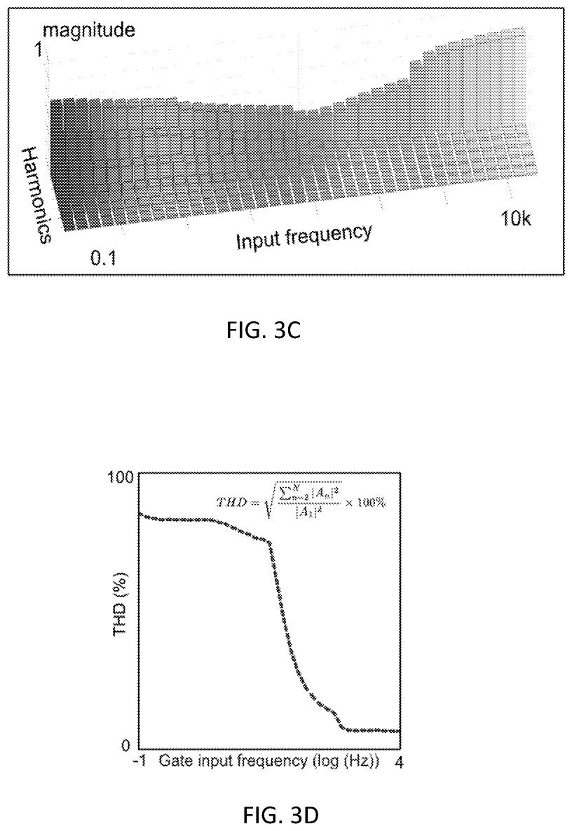

FIG. 3A shows time-domain representation of the input voltage (V_{g}, top) and corresponding output current (I_{ds}, bottom) and their use for detecting biomarkers at different input frequencies (0.1 Hz to 10 kHz). FIG. 3B show frequency-domain representation of the corresponding output current I_{ds} using FFT, revealing the harmonic components associated with each input frequency. FIG. 3C shows frequency-dependent response in ETRS for a fixed bioanalyte concentration, plotting the magnitude and phase of the I_{ds} across input frequencies from 0.1 Hz to 10 kHz, illustrating how higher-order harmonics enhance the fidelity of the measured signal. FIG. 3D shows the THD calculated at different input frequencies, showing how THD varies with frequency input, indicating the distortion levels in the ECT output current.

Mechanism-wise, ETRS employs a nonlinear detection method to analyze high-order harmonics of the electrochemical signals, offering deeper insights into the dynamics of the electrochemical double layer. This makes it a powerful technique for molecular detection. The embedded OECTs, with their high transconductance, further enhance the process by amplifying electrochemical signals and significantly improving the SNR.

Significantly, it is observed that a resonance frequency exists for each tested biosensor, corresponding to the highest sensitivity, which is up to six orders of magnitude higher than at other reference frequencies. This resonance indicates that an optimal synergy is reached between the detecting electrodes and the transistor, whose characteristics are both frequency-dependent. The sensor-specific resonance frequency can be easily identified by performing a full ETRS scan.

In ETRS analysis, the biosensor is treated as a unified, nonlinear “black box” system. For a given sensor, the gate sensing electrode and OECT transconductance may exhibit different frequency dependencies. However, isolating their individual contributions is unnecessary. Instead, the system is treated holistically, focusing on its overall frequency response. Once the system's inherent resonance frequency is determined through a simple pre-conditioned frequency scanning process, the lowest LoD can be correspondingly achieved by operating the biosensor at the resonance frequency.

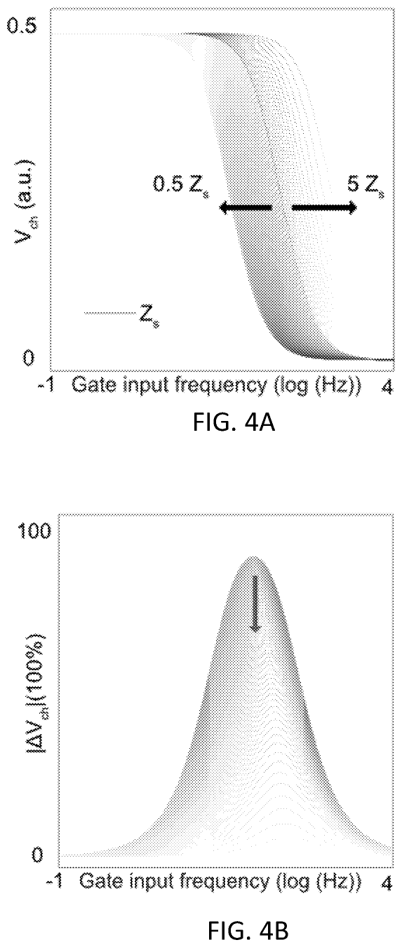

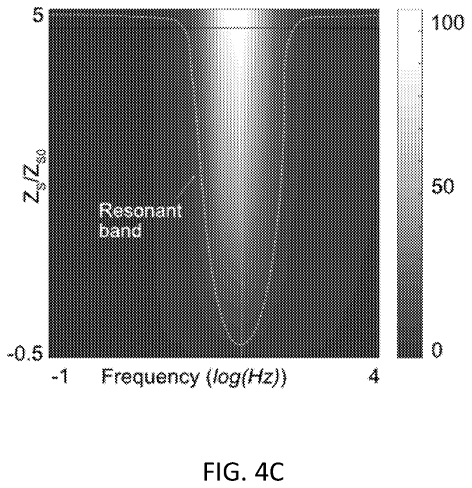

FIGS. 4A to 4C show analytical simulation results indicating the existence of the resonance frequency in the ETRS, which includes the absolute (FIG. 4A) and relative change (FIG. 4B) of voltage drop on channel with the increased input frequency. In short, under the resonance frequency, concentration changes of the biomarker cause substantial changes in the voltage drop (ΔV_{ch}) on the channel, correspondingly pushing the LoD in ETRS (FIG. 4C).

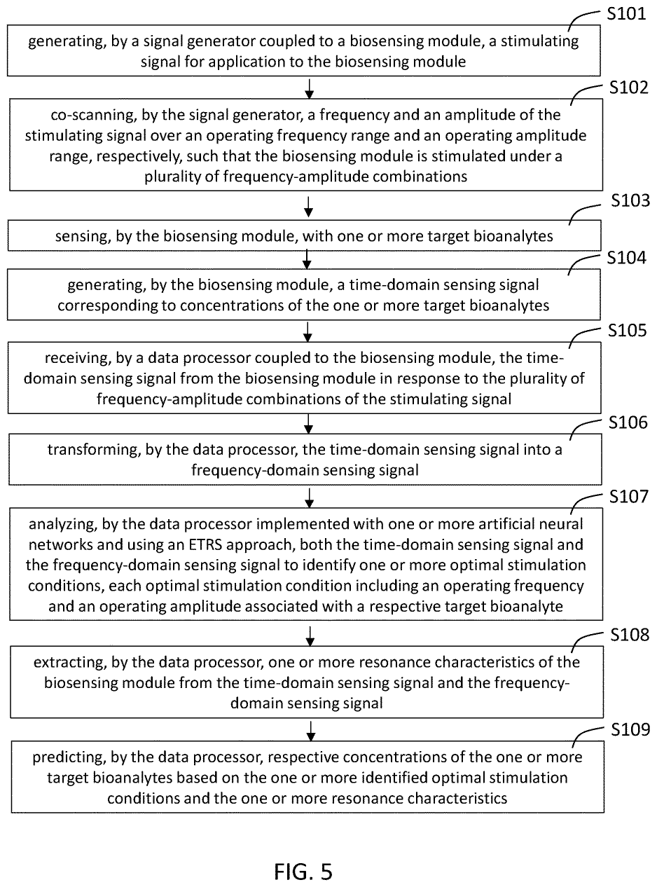

FIG. 5 illustrates a process flowchart of an ETRS-based OECT biosensing method for monitoring concentrations of one or more bioanalytes in accordance with the present invention. The method comprises the following steps:
S101: generating, by a signal generator coupled to a biosensing module, a stimulating signal for application to the biosensing module;
S102: co-scanning, by the signal generator, a frequency and an amplitude of the stimulating signal over an operating frequency range and an operating amplitude range, respectively, such that the biosensing module is stimulated under a plurality of frequency-amplitude combinations;
S103: sensing, by the biosensing module, with one or more target bioanalytes;
S104: generating, by the biosensing module, a time-domain sensing signal corresponding to concentrations of the one or more target bioanalytes;
S105: receiving, by a data processor coupled to the biosensing module, the time-domain sensing signal from the biosensing module in response to the plurality of frequency-amplitude combinations of the stimulating signal;
S106: transforming, by the data processor, the time-domain sensing signal into a frequency-domain sensing signal;
S107: analyzing, by the data processor implemented with one or more artificial neural networks (ANN) and using an ETRS approach, both the time-domain sensing signal and the frequency-domain sensing signal to identify one or more optimal stimulation conditions, each optimal stimulation condition including an operating frequency and an operating amplitude associated with a respective target bioanalyte;
S108: extracting, by the data processor, one or more resonance characteristics of the biosensing module from the time-domain sensing signal and the frequency-domain sensing signal; and
S109: predicting, by the data processor, respective concentrations of the one or more target bioanalytes based on the one or more identified optimal stimulation conditions and the one or more resonance characteristics.

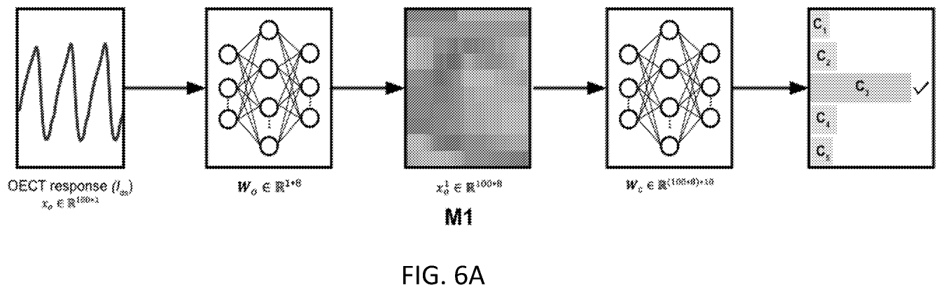

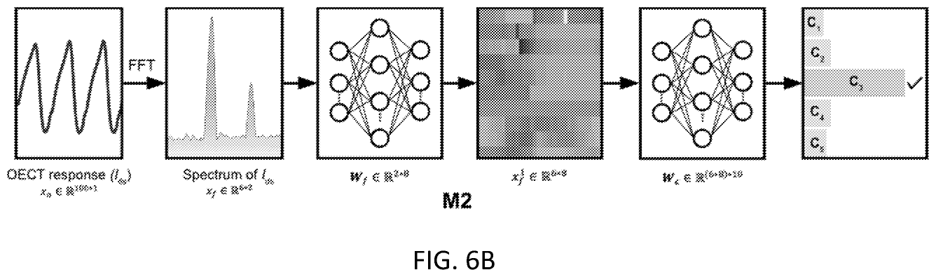

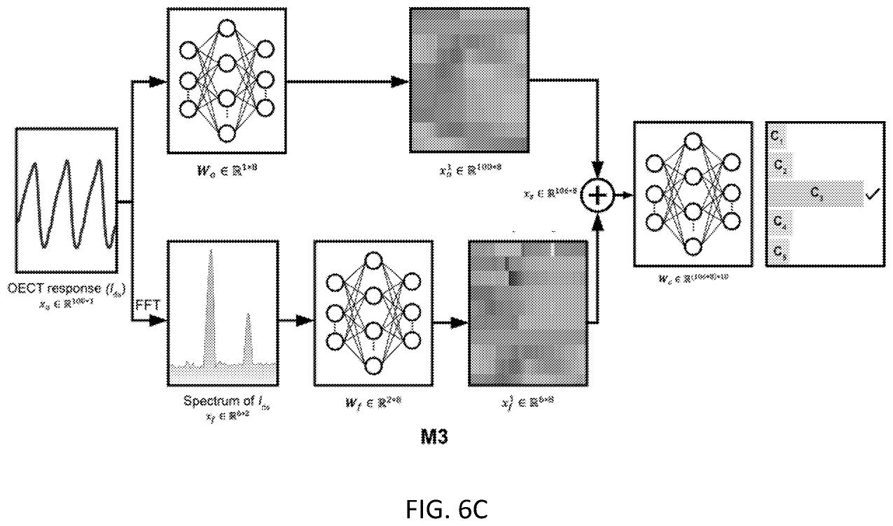

FIGS. 6A to 6C illustrates three different ANN architectures used for facilitating pattern recognition for the nonlinear sensing signal analysis in ETRS. FIG. 6A shows a non-FFT approach (M1), which can direct input of time-domain current signal; FIG. 6B shows a FFT approach (M2), which can input harmonic information derived from FFT of the time-domain signal; and FIG. 6C shows a combined approach (M3), which used an architecture combining both time-domain signal and FFT-derived harmonic information.

In the non-FFT approach M1, the time-domain sensing signal x₀ is analyzed using a first linear regression model network W₀ to obtain a set of time-domain feature embeddings

$${x}_{0}^{1} .$$

The time-domain feature embeddings

$${x}_{0}^{1}$$

is analyzed using a linear classifier W_{c} to obtain a set of candidate predictions Cᵢ. A final prediction (e.g., C₃) is identified from the set of candidate predictions Cᵢ.

Specifically, the first linear regression model network W₀ is trained/configured to analyze the time-domain sensing signal by: extracting resonance characteristics in the time-domain sensing signal as the time-domain feature embeddings

$${x}_{0}^{1} ;$$

and quantitively correlating the resonance characteristics with concentrations of the one or more target bioanalytes to obtain the one or more corresponding optimal stimulation voltages as the candidate predictions Cᵢ. Each optimal stimulation voltage is identified by locating an optimal frequence and an optimal amplitude of the stimulating signal at which a highest sensitivity for the corresponding target bioanalyte is achieved. The resonance characteristics include at least one of a resonance frequency, a resonance amplitude, a phase response, a bandwidth, or a quality factor.

In the FFT approach M2, the time-domain sensing signal x₀ is converted into a frequency-domain sensing signal x_{f} by performing a Fast Fourier Transform (FFT). The frequency-domain sensing signal is analyzed using a second linear regression model network W_{f} to obtain a set of frequency-domain feature embeddings

$${x}_{f}^{1} .$$

The frequency-domain feature embeddings

$${x}_{f}^{1}$$

is analyzed using a linear classifier W_{c} to obtain a set of candidate predictions Cᵢ. A final prediction (e.g., C₃) is identified from the set of candidate predictions Cᵢ.

In the combined approach M3, the time-domain sensing signal is converted into a frequency-domain sensing signal by performing FFT. The time-domain sensing signal is analyzed using a first linear regression model network LR1 to obtain a set of time-domain feature embeddings

$${x}_{0}^{1} .$$

The frequency-domain sensing signal is analyzed using a second linear regression model network LR2 to obtain a set of time-domain feature embeddings

$${x}_{f}^{1} .$$

The time-domain feature embeddings

$${x}_{0}^{1}$$

and time-domain feature embeddings

$${x}_{f}^{1}$$

are combined to obtain a set of combined feature embeddings x_{z}. The combined feature embeddings x_{z} is analyzed using a linear classifier LC to obtain a set of candidate predictions Cᵢ. A final prediction (e.g., C₃) is identified from the set of candidate predictions Cᵢ.

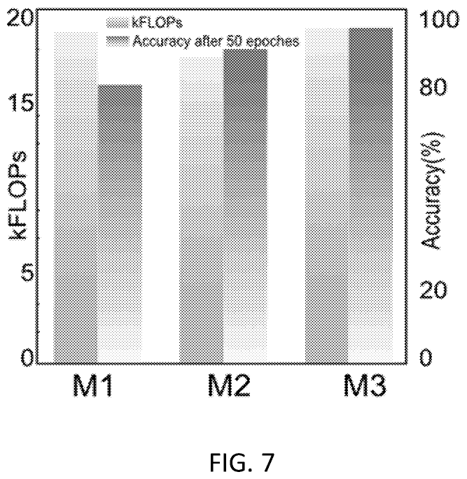

FIG. 7 shows comparison of computational efficiency and accuracy among the three approaches. The results demonstrate that, compared with the M1 and M2 approaches, the combined approach M3 can achieve the highest accuracy, up to 96%.

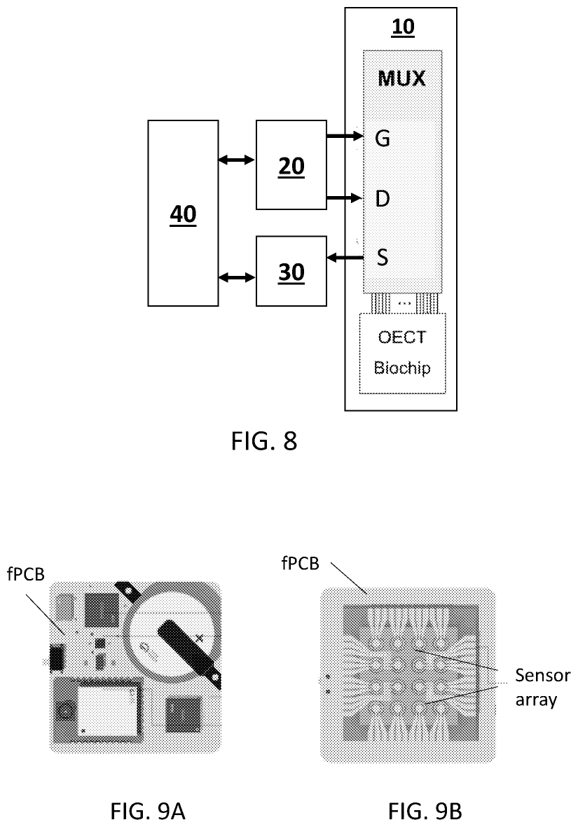

FIG. 8 illustrates a schematic diagram of a ETRS-based OECT biosensing system 1 for performing the method S100 in accordance with one embodiment of the present invention.

As shown, the ETRS-based OECT biosensing system 1 comprising a biosensing module 10 configured to sense one or more target bioanalytes and generate a time-domain sensing signal corresponding to concentrations of the one or more target bioanalytes, a signal generator 20 coupled to the biosensing module and configured to generate a stimulating signal for application to the biosensing module, and further configured to co-scan a frequency and an amplitude of the stimulating signal over an operating frequency range and an operating amplitude range, respectively, such that the biosensing module is stimulated under a plurality of frequency-amplitude combinations; a data processor 30 coupled to the biosensing module and configured to receive and process the time-domain sensing signal to estimate the concentrations of the one or more target bioanalytes.

The data processor 30 is implemented with one or more artificial neural networks (ANNs) and is configured to: receive the time-domain sensing signal from the biosensing module in response to the plurality of frequency-amplitude combinations of the stimulating signal; transform the time-domain sensing signal into a frequency-domain sensing signal; analyze, using the one or more artificial neural networks and an ETRS approach, both the time-domain sensing signal and the frequency-domain sensing signal to identify one or more optimal stimulation conditions, each optimal stimulation condition including an operating frequency and an operating amplitude associated with a respective target bioanalyte; extract one or more resonance characteristics of the biosensing module from the time-domain sensing signal and the frequency-domain sensing signal, the one or more resonance characteristics; and predict respective concentrations of the one or more target bioanalytes based on the one or more identified optimal stimulation conditions and the one or more resonance characteristics.

The biosensing module 10 comprises an OECT biochip and a multiplexer MUX electrically coupled to the OECT biochip. The OECT biochip includes one or more OECT biosensors for monitoring the one or more target bioanalytes respectively. The OECT biosensors may be arranged in an array of biosensing channels (e.g. 16 OECT biosensors arranged in a 4×4 array of biosensing channels).

Upon application of the stimulating signal to the biosensing module, each OECT biosensor in the biosensing module is driven by a gate-stimulating voltage V_{gs} to generate an enhanced drain-source current I_{ds} indicative of a concentration of the target bioanalyte sensed by the OECT biosensor.

Moreover, the amplitude and frequency of the gate-source voltage V_{gs} applied on each OECT biosensor are optimized to maximize affinity efficiency of bioreceptor for the corresponding target bioanalyte through the ETRS technique. The exact V_{gs} amplitude and the corresponding optimal frequency, referred to as the resonance frequency, can be determined by co-scanning both the amplitude and the frequency of the gate-source voltage V_{gs}.

At low frequencies, capacitive elements are nearly open-circuited, thus, the majority of the gate voltage is dropped on the channel, which is insensitive to the gate sensing process. At high frequencies, capacitive elements are nearly short-circuited, resulting in minimal voltage drop across the channel and weakening the amplification process. In contrast, at the resonance frequency, a maximum synergistic process is established between the sensing and amplification processes. Concentration changes of the biomarker cause substantial changes in the voltage drop on the channel regions, correspondingly pushing the LoD in ETRS, thus leading to the lowest LoD.

The biosensing system 1 may further include a wireless communication module 40 coupled to the signal generator 20 and the data processor 30, and configured to enable fully wireless data acquisition and transmission. The wireless communication module 40 may comprise a battery and Bluetooth Low Energy (BLE) chip. The battery may be a coin battery or a rechargeable battery. The wireless communication module may further comprise a power management unit, such as a low-dropout linear voltage regulator, which is connected to the battery and effectively regulates and supplies a stable voltage output to the BLE chip. For wireless data transmission, the BLE system-on-a-chip uses a miniature ceramic antenna operating at 2.45 GHz.

FIGS. 9A and 9B illustrate a top view and a bottom view of a prototype of the biosensing system 1, respectively. To further enhance comfort and wearability, a multilayer foldable flexible printed circuit board (fPCB) fabricated on flexible polyimide (PI) substrates may be employed to connect the electronic components and the OECT sensor array, distributing components across different sides to simplify connections between the sensor and the readout system. This stacking strategy minimizes the system's width and length, optimizing its physical dimensions and conformability.

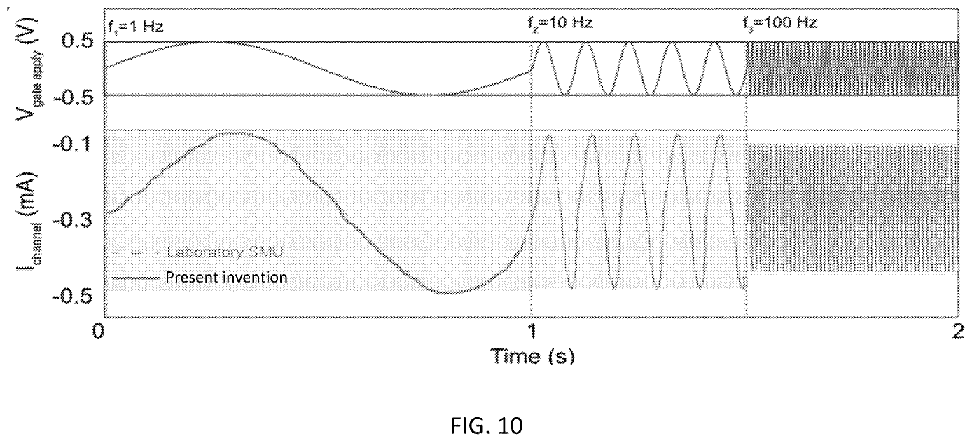

FIG. 10 illustrates experimental data that shows voltage input and current measurement at different frequencies. This demonstrates the system's capability to generate frequencies ranging from 0.1 Hz to 10 kHz and its high current measurement resolution (down to 1 nA,), which is comparable to the laboratory-use SMU.

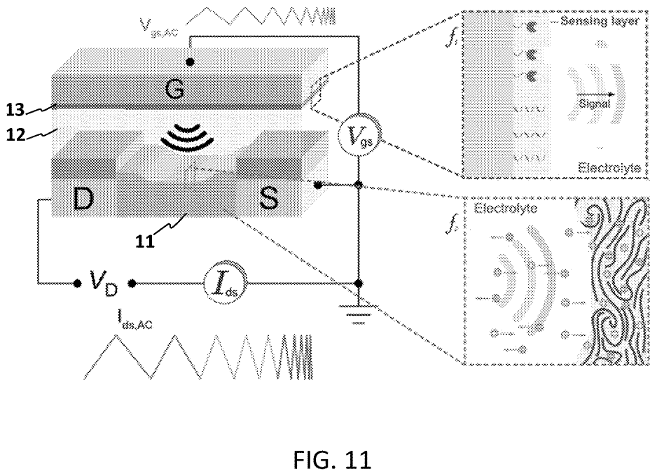

FIG. 11 illustrates a schematic diagram of an OECT biosensor in accordance with one embodiment of the present invention. The OECT biosensor comprises: a gate electrode G, a source electrode S, a drain electrode D, and an organic channel 11 disposed between the source electrode S and the drain electrode D.

The channel 11 is formed of an organic conducting material and is in contact with an electrolyte 12. A sensing layer 13 is deposited on the gate electrode G and arranged at an interface between the gate electrode G and the electrolyte 12, and is configured to interact with a target bioanalyte to modulate ionic transport within the electrolyte. The sensing layer 13 on the gate electrode G may be modified with a specific bioreceptor to detect a target bioanalyte, which will be discussed in more detail latter.

The electrolyte 12 is positioned between the gate electrode G and the channel 11 and is configured to support ionic conduction. Upon application of a gate-source voltage V_{gs}, on the gate electrode G, ions within the electrolyte 12 migrate toward or away from the channel region 11. The migrated ions penetrate into or are extracted from the organic channel 11, thereby modulating the doping state of the organic channel.

The channel 11 is electrically coupled between the source electrode S and the drain electrode D, and is configured to conduct a drain-source current I_{ds} depending on concentration of a target bioanalyte interacted with the sensing layer 13.

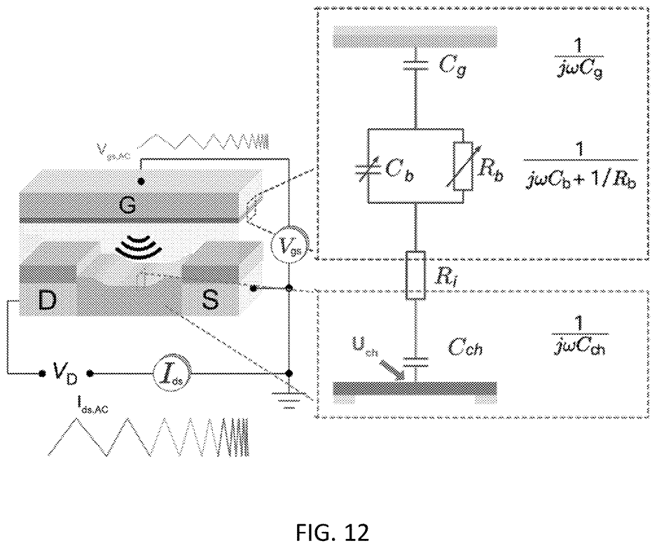

FIG. 12 illustrates impedance mathematical models for various circuit components of the OECT biosensor. The coupled mathematical model of the relationship between the voltage drop on the channel region (U_{ch}) with the entire impedance of equivalent circuit are as follows:

$${U}_{ch} = ( \frac{1}{j ω {C}_{ch}} ) / ( \frac{1}{j ω {C}_{g}} + \frac{1}{j ω {C}_{b} + \frac{1}{{R}_{b}}} + {R}_{i} + \frac{1}{j ω {C}_{ch}} )$$

where ω is the operation frequency in radian, C_{g} is the gate capacitance, C_{ch} is the channel capacitance, R_{b} and Cₚ are the equivalent resistance and capacitance of bulk electrolyte respectively, and Rᵢ is the equivalent resistance of the sensing layer.

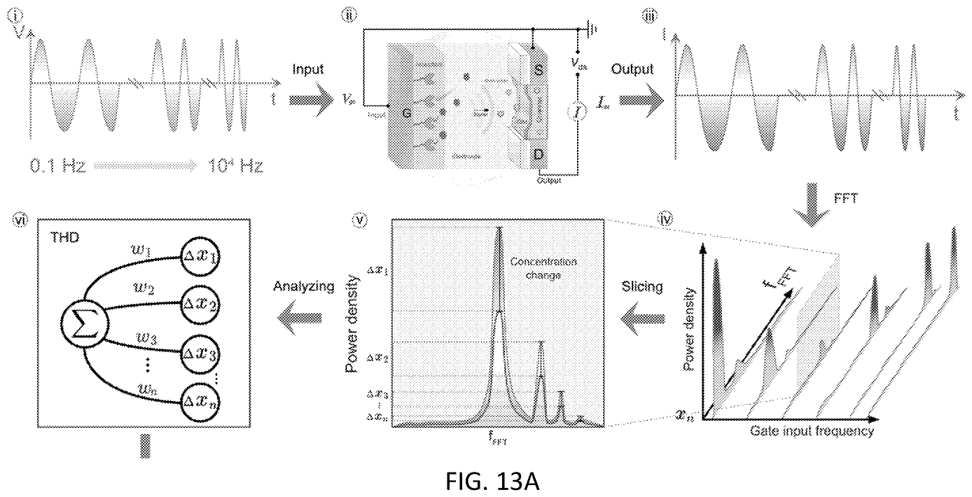

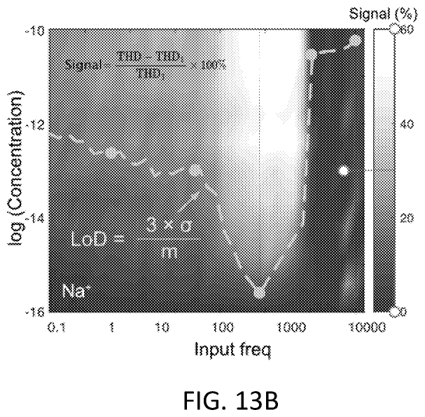

The operation of the ETRS-based OECT biosensor is detailed in FIG. 13A. Here, an OECT biosensor is used and modified with a bioreceptor for bioanalyte detection. (i) First, a large gate stimulating voltage V_{gs} (e.g., −0.5 V to 0.5 V) is applied to the gate (sensing) electrode of the OECT. (ii) The frequency of the gate stimulating voltage V_{gs} is then scanned from 0.1 Hz to 10 kHz to generate the entire ETRS spectrum and to locate the resonance frequency value. (iii) Next, the gate stimulating voltage V_{gs} is converted to an enhanced sensing current signal I_{ds} after being amplified by the OECT biosensor. (iv) The enhanced sensing current signal I_{ds} in the time domain is then transformed into the frequency domain through Fast Fourier transform (FFT). (v) Total harmonic distortion (THD) of the frequency-domain I_{ds} signals is analyzed at each input frequency and quantitatively correlated with concentration changes of the bioanalytes of interest, enabled by embedding an ANN.

FIG. 13B shows entire correspondingly generated ETRS spectrum (exemplified with Na⁺ ions) in which the resonance frequency can be easily identified (corresponding to the LoD indicated in the spectrum).

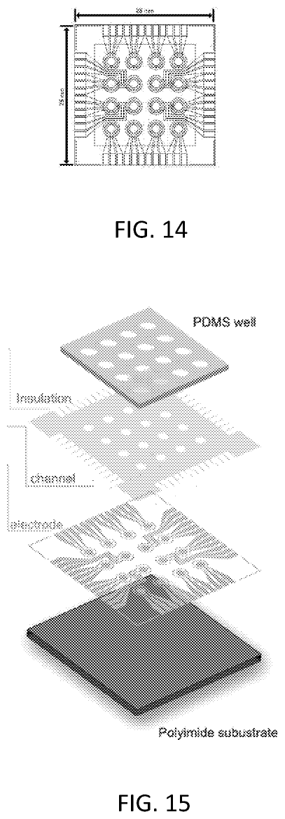

FIG. 14 is schematic depicting the layout of the OECT biochip, featuring an array of OECT sensor units. Each unit includes concentric electrode pads for the source(S), drain (D), and gate (G), with an active channel region measuring approximately 200 μm in width.

FIG. 15 is an exploded view of the biochip assembly, illustrating the sequential lamination of the electrode layer, channel layer (PEDOT:PSS), insulation layer, and the polydimethylsiloxane (PDMS) well atop a flexible polyimide substrate.

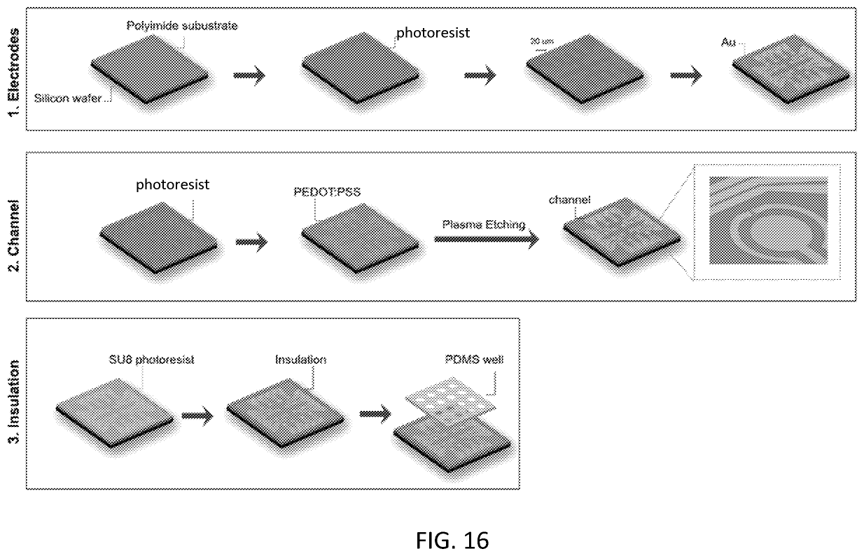

Referring to FIG. 16 for an example fabrication flow: 1) the electrodes are patterned by photolithography and deposited with gold on the polyimide substrate; 2) poly(3,4-ethylenedioxythiophene):polystyrene sulfonate (PEDOT:PSS) is subsequently spin-coated and etched to define channels; and (3) SU8 photoresist is used for electrical insulation, followed by integration of the PDMS well. The final panels show the assembled biochip arrays, ready for ETRS-based sensing measurements.

More specifically, the OECT biochip may be fabricated using multilayer photolithography technology. A flexible substrate was developed by casting a polyimide solution onto a silicon wafer. Then the source/drain/gate electrodes are constructed by depositing a layer of gold thin film (Cr, 10 nm; Au, 50 nm), with the pattern defined using a shadow mask.

Subsequently, the active channel layer is formed by spin-coating PEDOT:PSS on the device at 2000 rpm for 1 minute and then patterned through photolithography and plasma etching process.

The channel length and width of the devices were 20 and 200 μm, respectively. The PEDOT:PSS solution was prepared by firstly stirring for 3 min and then mixed with 3-glycidyloxypropyl trimethoxy silane (GOPS, 1%, w/w), glycerol (5%, v/v), and dodecylbenzene sulfonic acid (DBSA, 0.1%, v/v) with a Vortex mixer to form a mixed suspension. The addition of glycerol was to increase the film conductivity. DBSA was added to facilitate the wetting property of films on substrates.

Before spin-coating, the mixed suspension was filtered with a polytetrafluoroethylene membrane (aperture size of 0.45 μm) to remove aggregates. Next, SU-8 photoresist was patterned and solidified on the gold electrodes, serving as an insulating layer to protect them from exposure to the aqueous electrolyte. Finally, a 3D-printed polydimethylsiloxane (PDMS) well was transfer-printed onto the device to define the sensing area.

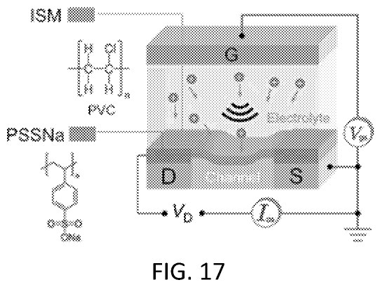

In some embodiments, as shown in FIG. 17, the OECT biosensor may be modified to be an ionic biosensor (e.g., a Na⁺ sensor) through incorporating an ion-selective membrane (ISM) and ionically conductive polymers (e.g., poly(sodium 4-styrenesulfonate), PSSNa) on the sensing layer to measure changes in ionic concentrations, thereby altering the OECT's gate response.

FIG. 18 illustrates steps for modification of an ISM on the channel of the OECT sensor to fabricate an OECT Na⁺ sensor. As shown, in step (1), the solution of PSSNa (1.2% w/v, molecular weight≈1000000 Da) was firstly spin-coated on the PEDOT:PSS channel at 1000 rpm, and then the polyelectrolyte film with GOPS was crosslinked by baking at 130° C. for 60 min and subsequently immersed in a 100 mM NaCl solution overnight to keep Nations in the film while removing any excess compounds. Finally, the ISM solution was drop-cast on top of the PSSNa film and dried at room temperature.

The Na⁺ ISM solution was prepared by combining Na⁺ ionophore X (1% w/w), bis(2-ethylhexyl) sebacate (DOS, 65.45% w/w), poly(vinyl chloride) (PVC, 33% w/w), and sodium tetrakis [3,5-bis(trifluoromethyl)phenyl]borate (Na-TFPB).

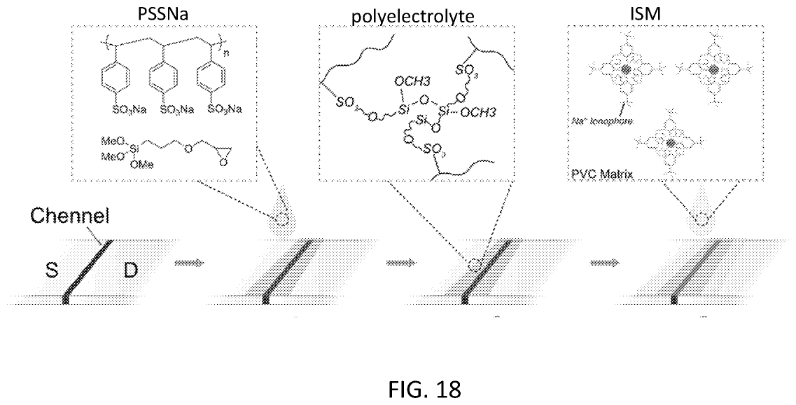

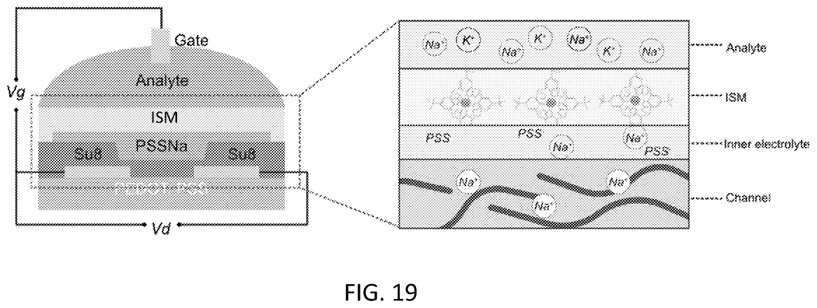

FIG. 19 shows a schematic structure of an ion selective OECT biosensor. Ion selectivity in the sensor is achieved by employing an ISM containing ionophores within a plasticized PVC matrix. To enhance functionality, bulky lipophilic ions, such as Na-TFPB (referred to as “ionic sites”), are embedded in the membrane. These ionic sites promote the exchange of target ions while preventing the passage of oppositely charged ions through the Donnan exclusion effect. DOS, a commonly used plasticizer, plays a crucial role in improving the mobility of charge carriers within the membrane. By combining these components, the resulting Na⁺ ISM achieves high ion selectivity and efficient charge transport.

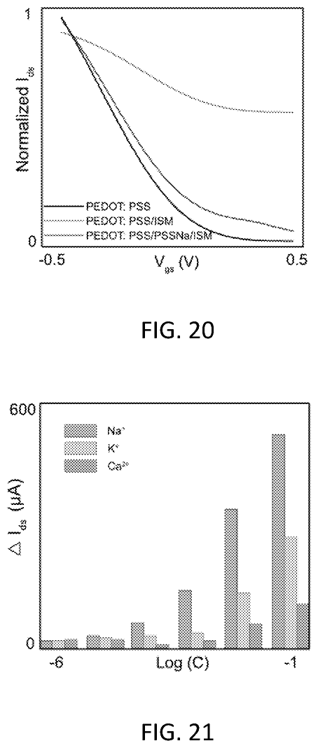

FIG. 20 shows transfer characteristics of a conventional OECT before and after coating an ISM, and the device with PSSNa as the inner electrolyte. The employment of inner electrolyte improves the on/off ratio of the OECT devices. FIG. 21 shows comparison of current response to Na, K⁺ and Ca²⁺ at different concentration at fixed V_{g}=0 V and V_{ds}=−0.4 V.

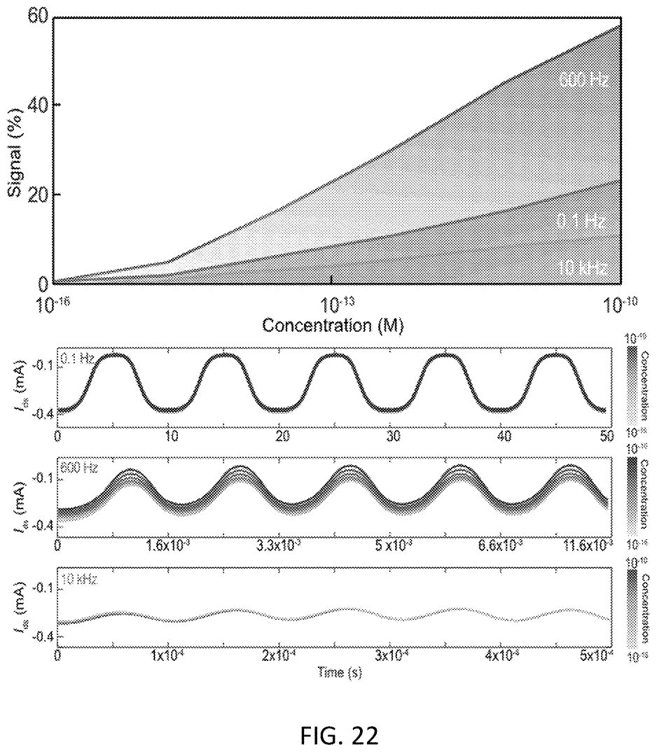

The efficacy of the ETRS-based biosensor was experimentally validated by detecting low concentration ionic species, exemplified by Na⁺ ions. FIG. 22 shows signals extracted from ETRS spectrum for sensing Na⁺ ions at different frequencies (0.1 Hz, 600 Hz, 10 kHz), which shows superior sensitivity at the resonance frequency (600 Hz).

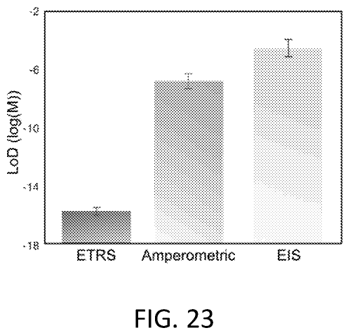

FIG. 23 illustrates the LoD of the ETRS-based biosensor for sensing Na⁺ ions over other competing technologies. The ETRS sensor achieved the best LoD of 10 fM at a resonance frequency of 600 Hz, which is 6 orders of magnitude lower than traditional EC and ECT sensors.

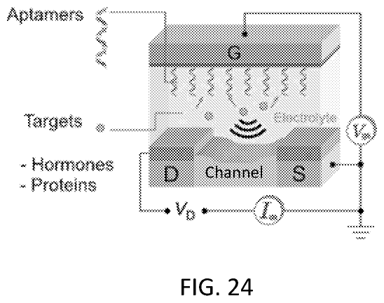

In one embodiment, the OECT sensor may be modified (or functionalized) to be an aptamer-based biosensor as shown in FIG. 24 by utilizing aptamer strands anchored on the sensing layer to detect hormones or proteins via high-affinity binding. This conformational change shifts the electrochemical properties at the electrolyte-gate interface.

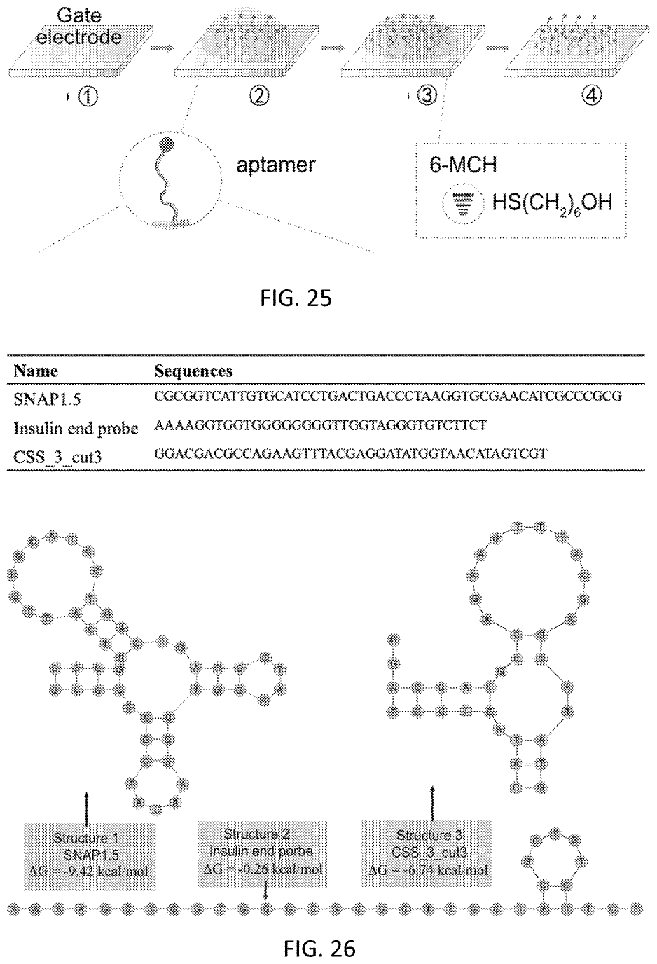

FIG. 25 is a schematic representation of the functionalization process, comprising four steps: (1) preparation of the electrode, (2) incubation with aptamer, (3) blocking with 6-mercaptohexanol (6-MCH), and (4) rinsing.

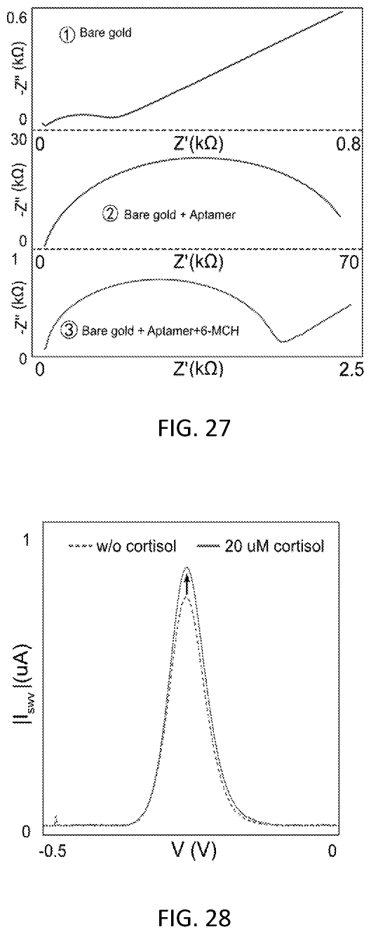

FIG. 26 illustrates sequences of the DNA aptamers used for target detection, including the insulin end probe and corresponding predicted 2D structures by Mfold. FIG. 27 illustrates impedance changes observed on the electrode surface throughout the functionalization process, illustrating the transition from bare gold to the aptamer-modified surface and subsequent blocking with 6-MCH. FIG. 28 shows verification of successful modification using Square Wave Voltammetry. The presence of a redox probe based on methylene blue on the aptamer allows for detection of target binding, as indicated by the shift in peak currents when comparing the absence of target (w/o cortisol) to the presence of 20 μM cortisol.

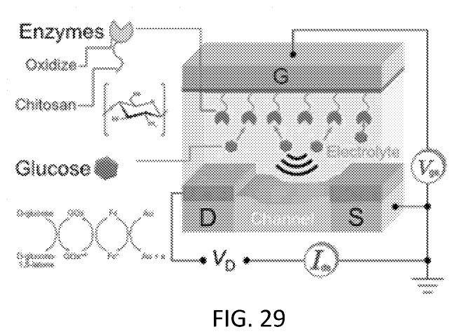

In one embodiment, the OECT biosensor may be modified (or functionalized) to be an enzyme-based OECT biosensor (e.g., a glucose biosensor) as shown in FIG. 29. Enzyme-based biosensing (teal) employs enzyme-functionalized (e.g., glucose oxidase) gate electrode (or sensing layer) surfaces to convert specific targets (such as glucose) into electroactive byproducts that modulate the channel current. More specifically, the functionalized sensing layer surface catalyzes a reaction involving a target bioanalyte to produce one or more electroactive species that modulate channel current.

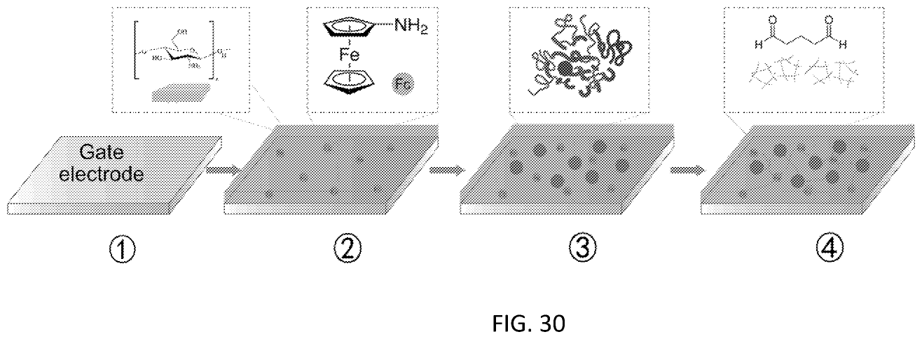

FIG. 30 shows steps for the preparation of enzyme-modified gate electrode of the OECT biosensor to form the enzyme-based OECT biosensor: 1) the gate electrode was firstly cleaned sequentially with deionized water, acetone, and ethanol, then dried at room temperature; 2) the mediator layer was modified on the electrode by dropping 10 μL of an aminoferrocene and chitosan mixture (prepared in phosphate-buffered saline (PBS) solution) and drying at room temperature; 3) the enzymatic layer was then formed by dropping 10 μL of a mixture containing glucose oxidase (500 U/mL) and chitosan, followed by drying at 4° C.; 4) finally, the composite enzymatic layer was crosslinked by applying 10 μL of a 0.3% (w/w) glutaraldehyde solution at 4° C. and allowing it to fully react for 48 hours.

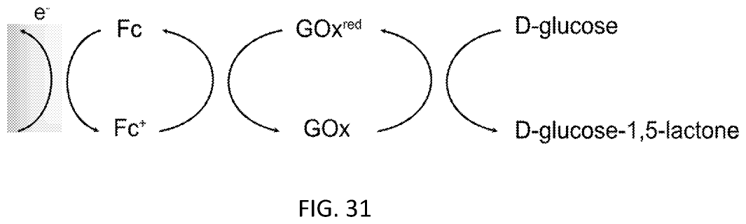

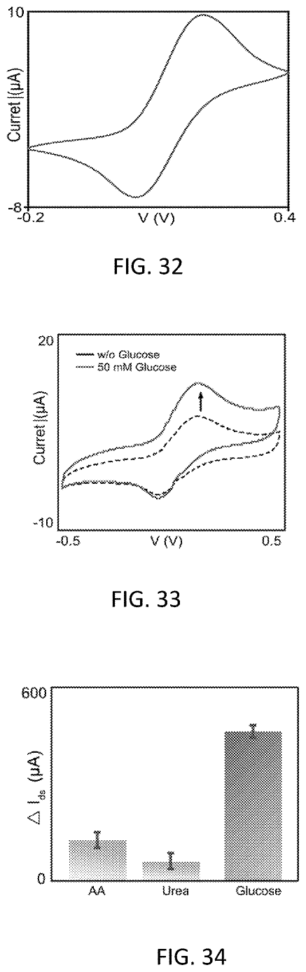

FIG. 31 shows the electron transfer process on an enzyme-modified electrode of a glucose biosensor in the presence of glucose. FIG. 32 shows cyclic voltammetry curve of the modified electrode in 0.1 M PBS. The presence of redox peak is ascribed to the ferrocene, which is the electron transfer mediator. FIG. 33 shows cyclic voltammetry curve of the modified electrode before and after adding 50 mM glucose. FIG. 34 shows response of OECT glucose sensors to potential interfering substances, including ascorbic acid (AA) and urea (n=5). Data are presented as means±SD.

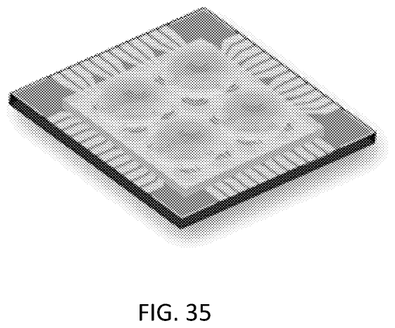

FIG. 35 shows an integrated ETRS-based OECT biosensing system integrated with an ionic biosensing unit, a enzymes-based biosensing unit, an aptamer-base biosensing unit and a cortisol sensing unit for detecting insulin, glucose, a virus protein (SARS-CoV-2 spike protein) and cortisol respectively.

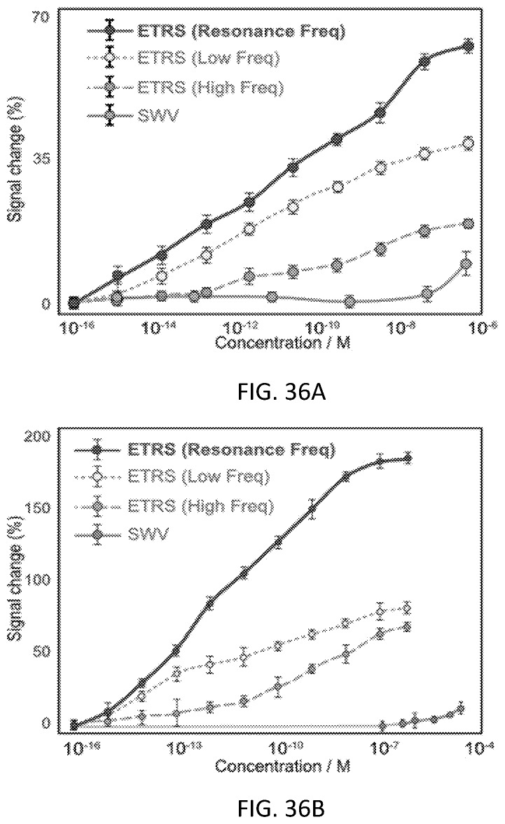

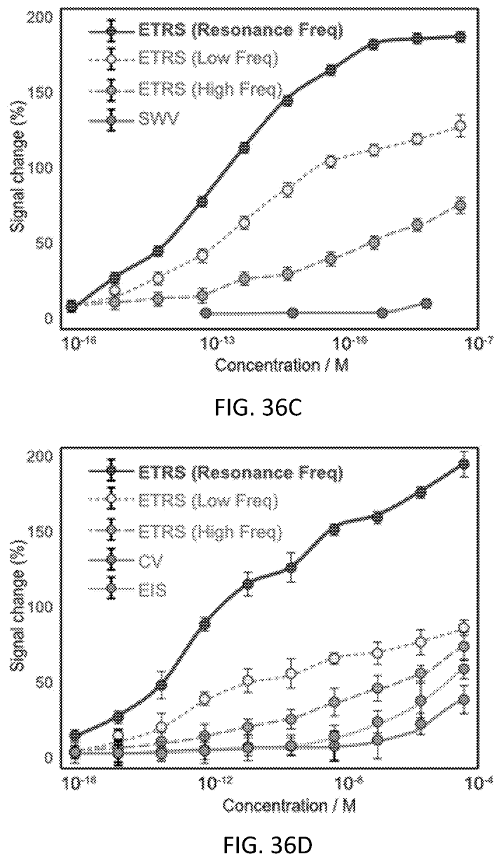

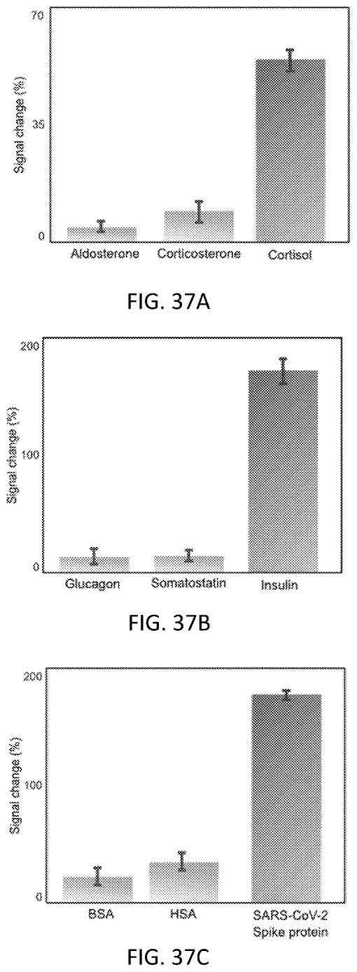

FIGS. 36A to 36D illustrate the improved sensitivity was further validated by comparing the LoD of different aptamer-based OECT biosensors detecting cortisol, insulin, and SARS-CoV-2 spike protein and glucose, respectively. All surpass the best results achievable with electrochemical biosensors (i.e. SWV, CV or EIS) respectively. The aptamer-OECT sensors maintained high signaling specificity in which insignificant signal change can be observed when each sensor was challenged against non-specific targets (FIGS. 37A to 37C).

The unique resonance frequency was identified for each sensor (600 Hz, 800 Hz, 900 Hz, 500 Hz respectively). Specifically, the cortisol biosensor reached the LoD valley of 10 fM at a frequency of 600 Hz, 2 orders of magnitude lower than the benchmarking frequency (1 Hz). The insulin biosensor achieved an LoD valley of 1 fM at 800 Hz, 4 orders of magnitude lower than the benchmarking frequency. The spike protein biosensor reached the LoD valley of 0.1 μM at a frequency of 900 Hz, 6 orders of magnitude lower than the benchmarking frequency. The enzyme-based glucose biosensors reached the LoD valley of 1 fM at a frequency of 500 Hz, 3 orders of magnitude lower than the benchmarking frequency.

It is noted that the resonance frequency for all ETRS sensors is typically in the range of 1-1000 Hz. This is understandable because, at low frequencies below 1 Hz, longer measurement times are required, making it challenging to establish a stable testing environment for analysis. On the other hand, at high frequencies above 1 kHz, the Gm of OECTs decreases dramatically, weakening the amplification ability.

The presented ETRS technique offers immediate applications to advance current electrochemical methods for detecting low-concentration bioanalytes. Among various scenarios, wearable sensors for detecting disease-related biomarkers are rapidly emerging. These devices provide continuous insights into individual health and performance while serving as key tools for collecting datasets essential to building large-scale health models, enabling digital health and AI-driven medicine.

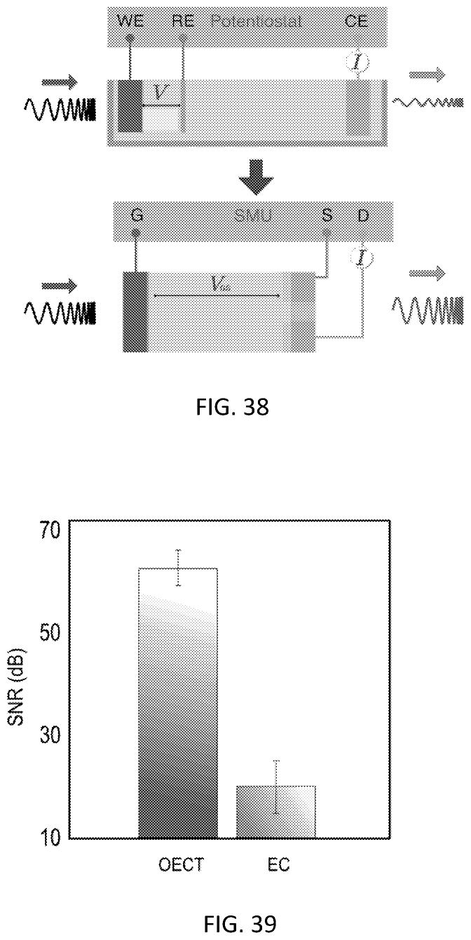

FIG. 38 shows characterization schematics of OECT device of the present invention and a conventional EC device. FIG. 39 shows SNR comparison under simulated noise conditions, where

$$SNR = 20 {log}_{10} ( \frac{\vert {I}_{signal} \vert}{\vert {I}_{noise} \vert} ) .$$

Notably, under the same stimulation voltage patterns, the responsive current of the ETRS sensor is 3 orders of magnitude higher than that of the reference EIS sensors, contributing to its high SNR and exceptionally low LoD.

To enable wearable applications, a compact and lightweight ETRS readout system is essential. A miniaturized ETRS readout system is provided in accordance with one embodiment of the present invention. In one example implementation, the miniaturized ETRS readout system has dimensions of 3.5 cm×3.5 cm×1.0 cm and a weight of only 2.5 g. This compact design allows seamless integration with smart wearables.

The presented wearable ETRS platform enables a wide range of applications for detecting low-concentration bioanalytes. As an example, we demonstrate its potential for wearable applications by detecting cortisol, a mental-health-related biomarker. Cortisol, with concentration as low as 1 nM, can be easily accessed from bodily fluids such as sweat. To ensure intimate contact between the sensor and the skin, the OECTs are fabricated on flexible polyimide, which conforms well to the skin. The gate electrodes (made of gold) of the flexible OECTs were modified with an aptamer designed to detect cortisol.

The gate electrode surface was cleaned before aptamer immobilization. This was accomplished by first rinsing the gold surface with ethanol and water. Then it was treated with piranha solution (H₂SO₄:H₂O₂=3:1) for 5 minutes, followed by prolonged rinsing in water. The treated surface was further incubated in 0.5 M H₂SO₄ for at least 30 minutes. The device was rinsed again with water and air dried. 5 μl of reduced aptamer solution was dropped onto the gold surface for immobilization overnight via self-assembled Au—S bond. The surface was rinsed with plenty of water to remove unbound aptamers. After drying, the aptamer monolayer was backfilled by incubating with 5 μl of 6 mM mercaptohexanol (MCH) for 3 hours. All incubation steps were conducted in room temperature with humidity control to prevent solution drying. The functionalized gate electrodes were rinsed in water and stored in 1×PBS in 4° C. before use.

The OECT sensors demonstrated stable performance under different bending conditions, ensuring their reliability for wearable sweat analysis. These flexible OECT sensors were connected to the ETRS readout system using a back-to-back attachment method. Silver paste was applied to solidify the connections and prevent disconnection during use.

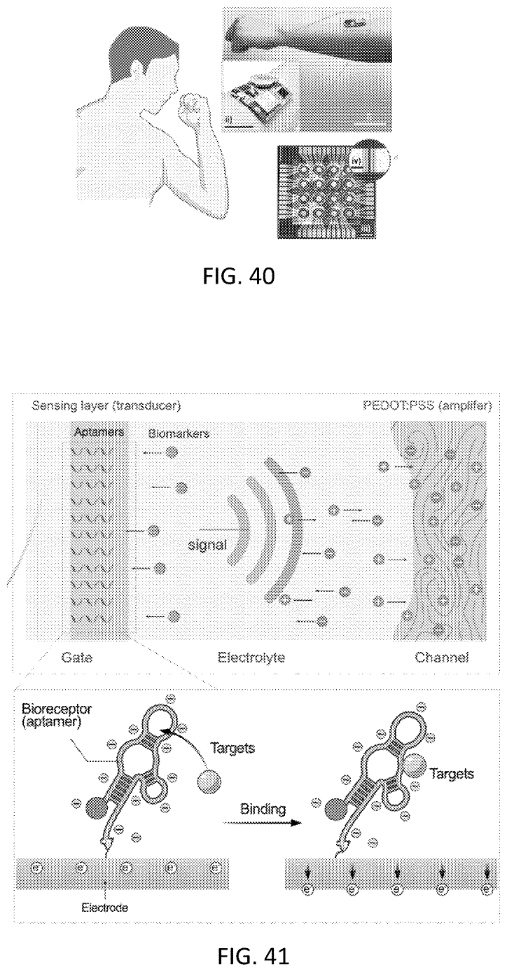

FIG. 40 shows an example implementation of an integrated wearable ETRS platform for the detection of low-concentration bioanalytes. The device is placed on the user's forearm for sweat-based measurements, which comprises a multichannel OECT sensor array and a wearable readout system.

FIG. 41 illustrates the sensing mechanism of the OECT biosensor. Target molecules (e.g., cortisol) are captured by the aptamer, and the signals are amplified by the OECT.

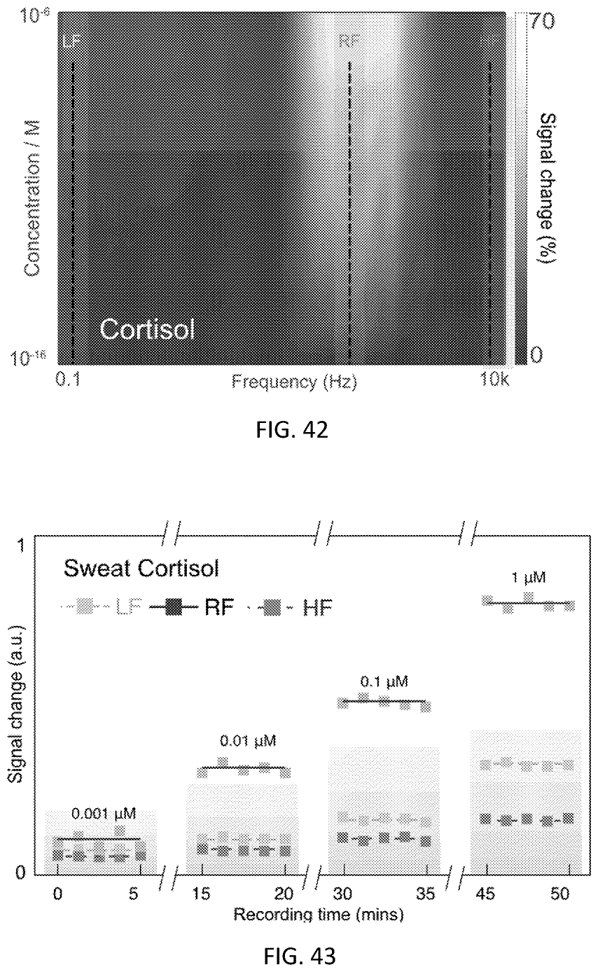

FIG. 42 shows an ETRS spectrum generated after a frequency scan, and the resonance frequency (RF) is correspondingly identified, where LF stands for low frequency (0.1 Hz) and HF stands for high frequency (10 kHz). For wearable measurements, we first performed a full-frequency scan of the ETRS to assess sensitivity across different frequencies and identify the resonance frequency. In this experiment, a resonance frequency of 600 Hz was identified, corresponding to the lowest LoD and highest sensitivity. This frequency was then selected for all subsequent experiments.

FIG. 43 shows experimental data of the cortisol concentration signals extracted from the ETRS spectrum under LF, RF and HF. The result shows the superior sensitivity of the OECT sensors operated at RF with the LoD lower that 1 nM that well covers the concentration range of sweat cortisol.

Referring to FIGS. 42 and 43, the wearable ETRS platform accurately monitors cortisol concentration in sweat, detecting a range from 1 nM to 1 μM, validated against a reference device. When operated at the resonance frequency (600 Hz), the ETRS demonstrated the highest sensitivity, capable of detecting concentration changes lower than 1 nM. This sensitivity was not achievable at other reference frequencies (e.g., 0.1 Hz or 10 kHz), confirming its viability in real-world wearable scenarios.

It should be understood that the application of ETRS is not limited to wearable scenarios and can be extended to any context where electrochemical sensors are used. For example, facilitated by our accessible readout system, ETRS can enhance the sensitivity of biochips for multimodal sensing.

The functional units and modules in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

While the present disclosure has been described and illustrated with reference to specific embodiments thereof, these descriptions and illustrations are not limiting. The illustrations may not necessarily be drawn to scale. There may be distinctions between the illustrations in the present disclosure and the actual apparatus due to manufacturing processes and tolerances. There may be other embodiments of the present disclosure which are not specifically illustrated. Modifications may be made to adapt a particular situation, material, composition of matter, method, or process to the objective and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. An ETRS-based biosensing system, comprising:
a biosensing module configured to sense one or more target bioanalytes and generate a time-domain sensing signal corresponding to concentrations of the one or more target bioanalytes;
a signal generator coupled to the biosensing module and configured to generate a stimulating signal for application to the biosensing module, and further configured to co-scan a frequency and an amplitude of the stimulating signal over an operating frequency range and an operating amplitude range, respectively, such that the biosensing module is stimulated under a plurality of frequency-amplitude combinations; and
a data processor coupled to the biosensing module and configured to receive and process the time-domain sensing signal to estimate the concentrations of the one or more target bioanalytes;
wherein the data processor is implemented with one or more artificial neural networks and is configured to:
receive the time-domain sensing signal from the biosensing module in response to the plurality of frequency-amplitude combinations of the stimulating signal;
transform the time-domain sensing signal into a frequency-domain sensing signal;
analyze, using the one or more artificial neural networks and an ETRS approach, both the time-domain sensing signal and the frequency-domain sensing signal to identify one or more optimal stimulation conditions, each optimal stimulation condition including an operating frequency and an operating amplitude associated with a respective target bioanalyte;
extract one or more resonance characteristics of the biosensing module from the time-domain sensing signal and the frequency-domain sensing signal, the one or more resonance characteristics; and
estimate the concentrations of the one or more target bioanalytes based on the one or more identified optimal stimulation conditions and the one or more resonance characteristics.

2. The ETRS-based biosensing system according to claim 1, wherein the biosensing module comprises an OECT biochip and a multiplexer electrically coupled to the OECT biochip, and wherein the OECT biochip includes one or more OECT biosensors configured to monitor the one or more target bioanalytes, respectively.

3. The ETRS-based biosensing system according to claim 2, wherein each OECT biosensor comprises:
a gate electrode;
a source electrode;
a drain electrode;
an organic channel disposed between the source electrode and the drain electrode;
an electrolyte positioned between the gate electrode and the organic channel; and
a sensing layer deposited on the gate electrode and arranged at an interface between the gate electrode and the electrolyte.

4. The ETRS-based biosensing system according to claim 3, wherein when the stimulating signal is applied to the biosensing module, each OECT biosensor is driven by a gate-stimulating voltage V_{gs} to generate an enhanced drain-source current I_{ds} indicative of a concentration of the target bioanalyte sensed by the OECT biosensor.

5. The ETRS-based biosensing system according to claim 4, wherein the OECT biochip includes an ionic biosensor having an ion-selective membrane and an ionically conductive polymer incorporated on a sensing layer of the ionic biosensor.

6. The ETRS-based biosensing system according to claim 4, wherein the OECT biochip includes an aptamer-based biosensor having aptamer strands anchored on a sensing layer of the aptamer-based biosensor to detect hormones or proteins via high-affinity binding.

7. The ETRS-based biosensing system according to claim 4, wherein the OECT biochip includes an enzyme-based OECT biosensor having a functionalized sensing layer surface configured to convert a specific target bioanalyte into electroactive byproducts that modulate channel current.

8. The ETRS-based biosensing system according to claim 4, wherein the OECT biochip includes an enzyme-based OECT biosensor having a functionalized sensing layer surface configured to catalyze a reaction involving a target bioanalyte to produce one or more electroactive species that modulate channel current.

9. The ETRS-based biosensing system according to claim 1, further comprising a wireless communication module coupled to the signal generator and the data processor, and configured to enable fully wireless data acquisition and transmission.

10. The ETRS-based biosensing system according to claim 1, wherein the one or more resonance characteristics include at least one of a resonance frequency, a resonance amplitude, a phase response, a bandwidth, or a quality factor.

11. An ETRS-based biosensing method, comprising:
generating, by a signal generator coupled to a biosensing module, a stimulating signal for application to the biosensing module;
co-scanning, by the signal generator, a frequency and an amplitude of the stimulating signal over an operating frequency range and an operating amplitude range, respectively, such that the biosensing module is stimulated under a plurality of frequency-amplitude combinations;
sensing, by the biosensing module, with one or more target bioanalytes;
generating, by the biosensing module, a time-domain sensing signal corresponding to concentrations of the one or more target bioanalytes;
receiving, by a data processor coupled to the biosensing module, the time-domain sensing signal from the biosensing module in response to the plurality of frequency-amplitude combinations of the stimulating signal;
transforming, by the data processor, the time-domain sensing signal into a frequency-domain sensing signal;
analyzing, by the data processor implemented with one or more artificial neural networks and using an ETRS approach, both the time-domain sensing signal and the frequency-domain sensing signal to identify one or more optimal stimulation conditions, each optimal stimulation condition including an operating frequency and an operating amplitude associated with a respective target bioanalyte;
extracting, by the data processor, one or more resonance characteristics of the biosensing module from the time-domain sensing signal and the frequency-domain sensing signal; and
predicting, by the data processor, respective concentrations of the one or more target bioanalytes based on the one or more identified optimal stimulation conditions and the one or more resonance characteristics.

12. The ETRS-based biosensing method according to claim 11, wherein the biosensing module comprises an OECT biochip and a multiplexer electrically coupled to the OECT biochip, and wherein the OECT biochip includes one or more OECT biosensors configured to monitor the one or more target bioanalytes, respectively.

13. The ETRS-based biosensing method according to claim 12, wherein each OECT biosensor comprises:
a gate electrode;
a source electrode;
a drain electrode;
an organic channel disposed between the source electrode and the drain electrode;
an electrolyte positioned between the gate electrode and the organic channel; and
a sensing layer deposited on the gate electrode and arranged at an interface between the gate electrode and the electrolyte.

14. The ETRS-based biosensing method according to claim 13, wherein when the stimulating signal is applied to the biosensing module, each OECT biosensor is driven by a gate-stimulating voltage V_{gs} to generate an enhanced drain-source current I_{ds} indicative of a concentration of the target bioanalyte sensed by the OECT biosensor.

15. The ETRS-based biosensing method according to claim 14, wherein the OECT biochip includes an ionic biosensor having an ion-selective membrane and an ionically conductive polymer incorporated on a sensing layer of the ionic biosensor.

16. The ETRS-based biosensing method according to claim 14, wherein the OECT biochip includes an aptamer-based biosensor having aptamer strands anchored on a sensing layer of the aptamer-based biosensor to detect hormones or proteins via high-affinity binding.

17. The ETRS-based biosensing method according to claim 14, wherein the OECT biochip includes an enzyme-based OECT biosensor having a functionalized sensing layer surface configured to convert a specific target bioanalyte into electroactive byproducts that modulate channel current.

18. The ETRS-based biosensing method according to claim 14, wherein the OECT biochip includes an enzyme-based OECT biosensor having a functionalized sensing layer surface configured to catalyze a reaction involving a target bioanalyte to produce one or more electroactive species that modulate channel current.

19. The ETRS-based biosensing method according to claim 11, further comprising enabling, by a wireless communication module coupled to the signal generator and the data processor, fully wireless data acquisition and transmission.

20. The ETRS-based biosensing method according to claim 11, wherein the one or more resonance characteristics include at least one of a resonance frequency, a resonance amplitude, a phase response, a bandwidth, or a quality factor.


SEQUENCE LISTING (3 sequences)

SEQ ID NO: 1 - DNA, 50 bp, synthetic construct
  source 1..50: mol_type = other DNA
cgcggtcattgtgcatcctgactgaccctaaggtgcgaacatcgcccgcg

SEQ ID NO: 2 - DNA, 35 bp, synthetic construct
  source 1..35: mol_type = other DNA
aaaaggtggtggggggggttggtagggtgtcttct

SEQ ID NO: 3 - DNA, 42 bp, synthetic construct
  source 1..42: mol_type = other DNA
ggacgacgccagaagtttacgaggatatggtaacatagtcgt